(19) United States
(12) Patent Application Publication
(10) Pub. No.: US 2006/0247524 A1
(43) Pub. Date: Nov. 2, 2006

(54) Diagnostic ultrasound system

(75) Inventors: Zhengyi Zhang, Beijing (CN); Shinichi Amemiya, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/409,690

(22) Filed: Apr. 24, 2006

(30) Foreign Application Priority Data
Apr. 30, 2005 (CN) 200510068996.0

(51) Int. Cl. A61B 8/00

(52) U.S. Cl. 600/437

(57) ABSTRACT

An object of the present invention is to provide a battery-driven ultrasound diagnostic apparatus in which the power consumption required for lighting keys little affects an operation time. An ultrasound diagnostic apparatus with lit operation keys comprises a power supply unit that switches power to be supplied to the components of the apparatus between power fed from a battery and power fed from the mains, and a lighting adjustor that ceases or minimizes lighting of operation keys when a detected ambient brightness level exceeds a predetermined threshold.

### BACKGROUND OF THE INVENTION

The present invention relates to an ultrasound diagnostic apparatus, or more particularly, to an ultrasound diagnostic apparatus with lit operation keys.

### Background Art

Operation keys in an ultrasound diagnostic apparatus are lighted from their back. This is intended to make the operation keys, which are included in the ultrasound diagnostic apparatus that is often used in a dim room, easier to see. A room is dimmed so that ultrasonic images displayed on a display unit can be seen easily (refer to, for example, Patent Document 1).

[Patent Document 1] Japanese Unexamined Patent Application Publication No. 2000-5174 (pp. 2 to 3, FIG. 1)

An ultrasound diagnostic apparatus designed to be compact and lightweight for better portability adopts a battery as a power supply. In the ultrasound diagnostic apparatus adopting the battery as the power supply, power to be consumed for lighting operation keys greatly affects an operation time.

### SUMMARY OF THE INVENTION

Therefore, an object of the present invention is to provide a battery-driven ultrasound diagnostic apparatus in which the power consumption required for lighting keys little affects an operation time.

The present invention attempting to accomplish the above object provides an ultrasound diagnostic apparatus with lit operation keys comprising a power supply means for switching power to be supplied to the components of the apparatus between power fed from a battery and power fed from the commercial power outlet (hereinafter, mains), and a lighting adjustment means for ceasing or minimizing lighting of the operation keys when a detected ambient brightness level exceeds a predetermined threshold.

Preferably, the lighting adjustment means lights each of the operation keys by flickering a light source at intervals of a time which is so short that the flicker is indiscernible with naked eyes, and detects an ambient brightness level at the cyclic timing of putting out the light source. Consequently, the ambient brightness level can be detected readily.

Preferably, the lighting adjustment means ceases or minimizes lighting of the keys when an ambient brightness level detected when power is fed from the battery exceeds a predetermined threshold. Consequently, lighting can be adjusted according to a type of power supply.

Preferably, the lighting adjustment means decreases the threshold, relative to which a brightness level is determined, along with a decrease in a remaining battery capacity. This contributes to extension of an operation time.

Preferably, when power is fed from the mains, the lighting adjustment means makes the threshold, relative to which a brightness level is determined, larger than when power is fed from the battery. This makes the operation keys easier to see.

Preferably, when power is fed from the mains, the lighting adjustment means lights the operation keys irrespective of an ambient brightness level. This makes the operation keys much easier to see.

According to the present invention, the ultrasound diagnostic apparatus with lit operation keys comprises the power supply means for switching power to be supplied to the components of the apparatus between power fed from a battery and power fed from the mains, and the lighting adjustment means for ceasing or minimizing lighting of the operation keys when a detected ambient brightness level exceeds a predetermined threshold. This signifies realization of a battery-driven ultrasound diagnostic apparatus in which the power consumption required for lighting keys little affects an operation time.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram of an ultrasound diagnostic apparatus that is an example of the best mode for implementing the present invention.

FIG. 2 is a conceptual diagram concerning scanning with a sound ray.

FIG. 3 is a conceptual diagram concerning scanning with a sound ray.

FIG. 4 is a conceptual diagram concerning scanning with a sound ray.

FIG. 5 schematically shows the appearance of the ultrasound diagnostic apparatus that is an example of the best mode for implementing the present invention.

FIG. 6 is a sectional view showing the structure of an operation key included in the ultrasound diagnostic apparatus that is an example of the best mode for implementing the present invention.

FIG. 7 is a block diagram showing the configuration of part of the ultrasound diagnostic apparatus that is an example of the best mode for implementing the present invention.

### DETAILED DESCRIPTION OF THE INVENTION

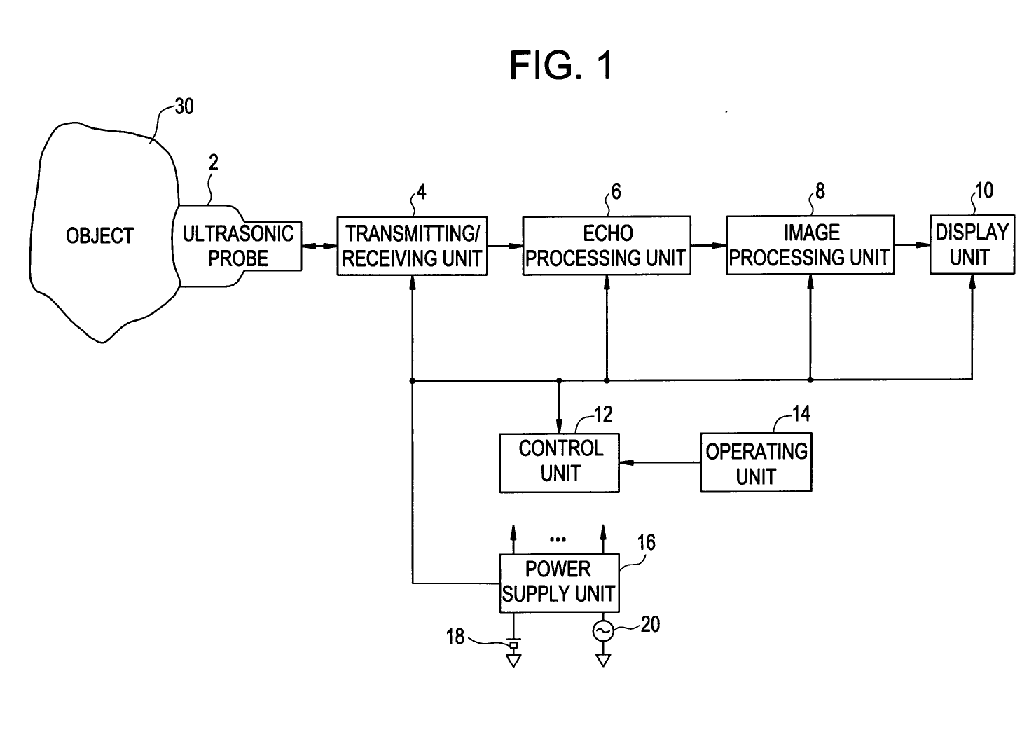

Referring to drawings, the best mode for implementing the invention will be described below. The present invention is not limited to the best mode for implementing the invention. FIG. 1 is a block diagram showing an ultrasound diagnostic apparatus. The present apparatus is an example of the best mode for implementing the invention. The configuration of the apparatus provides an example of the best mode for implementing the present invention in an ultrasound diagnostic apparatus.

As shown in FIG. 1, the ultrasound diagnostic apparatus includes an ultrasonic probe 2. The ultrasonic probe 2 comprises an ultrasonic transducer array. Ultrasonic transducers included in the ultrasonic transducer array are made of a piezoelectric material such as a PZT (titanium (Ti) zirconate (Zr) lead (Pb)) ceramic.

The ultrasonic probe 2 is brought into contact with an object 30 by a user. The ultrasonic probe 2 is connected to a transmitting/receiving unit 4. The transmitting/receiving unit 4 applies a driving signal to the ultrasonic probe 2 and thus causes the ultrasonic probe 2 to transmit an ultrasonic beam. The transmitting/receiving unit 4 receives echoes collected by the ultrasonic probe 2.

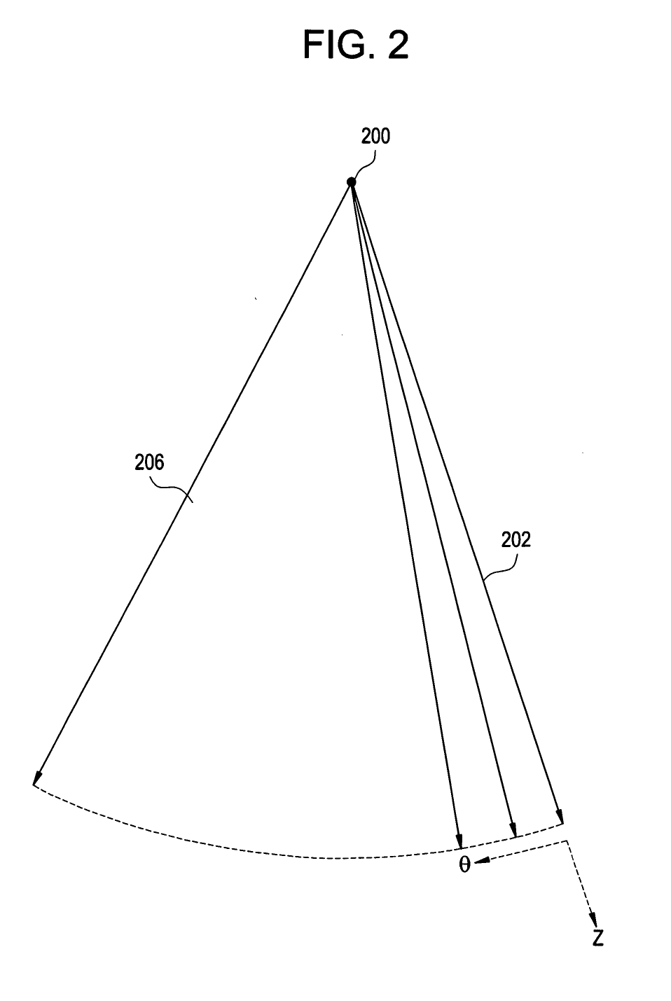

The transmitting/receiving unit 4 scans an object as shown in FIG. 2. Specifically, the transmitting/receiving unit performs so-called sector scanning so as to scan a sector-shaped two-dimensional region 206 in a θ direction using a sound ray 202 that extends from a radiating point 200 in a z direction. The sound ray corresponds to the ray axis of an ultrasonic beam.

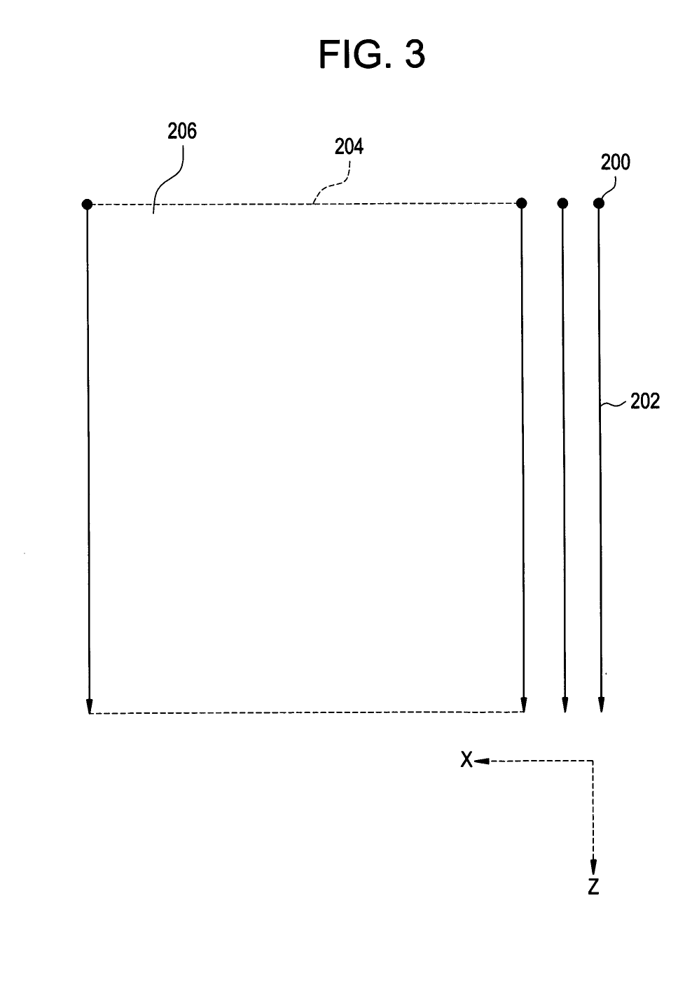

When an opening through which ultrasonic waves are transmitted or echoes are received is formed using part of the ultrasonic transducer array, an object can be scanned, for example, as shown in FIG. 3 by shifting the position of the opening along the array. Specifically, so-called linear scanning is performed in order to scan a rectangular two-dimensional region 206 in an x direction by changing the sound rays 202, which are radiated from the radiating point 200 in the z direction, from one to another in parallel along a linear trajectory 204.

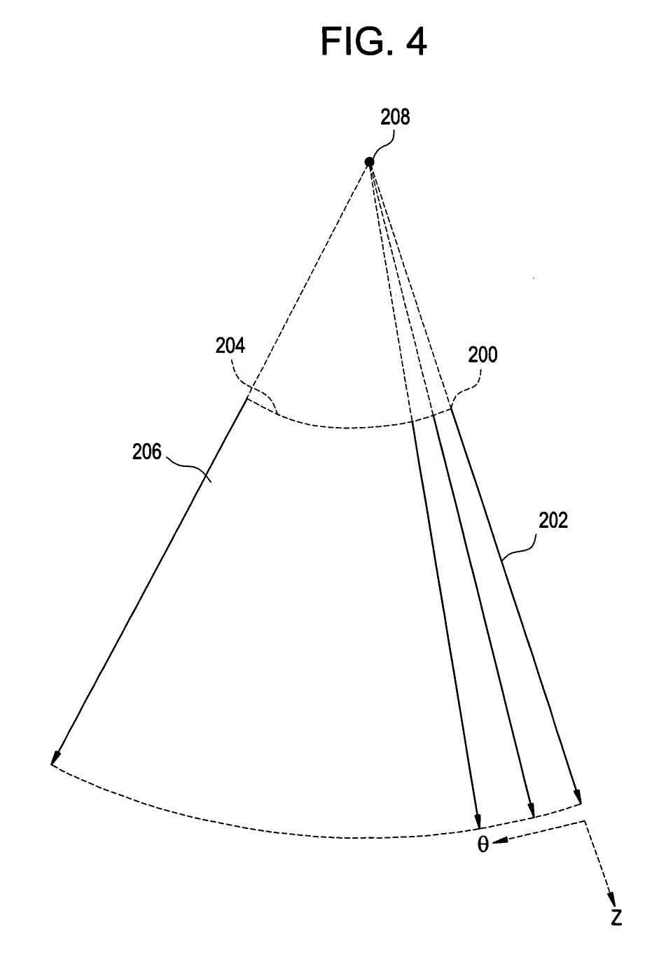

Incidentally, if the ultrasonic transducer array is a so-called convex array that swells out in an arc form in a direction in which ultrasonic waves are transmitted, scanning is performed by changing the sound rays from one to another in the same manner as those in the linear scan mode. Namely, for example, as shown in FIG. 4, so-called convex scanning is performed in order to scan a sector-shaped two-dimensional region 206 in a θ direction by shifting the radiating point 200, from which the sound ray 202 is radiated, along an arc-shaped trajectory 204.

The transmitting/receiving unit 4 is connected to an echo processing unit 6. Echoes received in response to each sound ray radiated from the transmitting/receiving unit 4 are transferred to the echo processing unit 6. The echo processing unit 6 processes echoes to produce an image signal associated with each sound ray.

The echo processing unit 6 is connected to an image processing unit 8. The image processing unit 8 constructs an image on the basis of image signals transferred from the echo processing unit 6. For construction of an image, the image processing unit 8 converts the layout of image signals associated with the layout of sound rays into a grid-like layout developed in a two-dimensional space by making the most of the capability of a scan converter thereof.

A display unit 10 is connected to the image processing unit 8. An image sent from the image processing unit 8 is displayed on the display unit 10. The display unit 10 is realized with a graphic display or the like.

A control unit 12 is connected to the foregoing transmitting/receiving unit 4, echo processing unit 6, image processing unit 8, and display unit 10. The control unit 12 applies a control signal to each of the units so as to control the actions of the units. Moreover, various notification signals are transferred from the controlled units to the control unit 12. Ultrasonic imaging is performed under the control of the control unit 12.

An operating unit 14 is connected to the control unit 12. The operating unit 14 is handled by a user, whereby an appropriate command or information is transferred to the control unit 12. The operating unit 14 includes, for example, a keyboard, a pointing device, and other operating tools.

The display unit 10 and operating unit 14 serve as a man-machine interface included in the ultrasound diagnostic apparatus. A user can interactively operate the apparatus via the man-machine interface.

A power supply means 16 supplies power to the foregoing units starting with the transmitting/receiving unit 4 and ending with the control unit 12. A power source is a battery 18 or the mains 20. The battery 18 is incorporated in the apparatus. The mains is, for example, an externally supplied commercial power outlet. One of the battery 18 and mains 20 is automatically or manually selected and put to use. The power supply means 16 is controlled by the control unit 12. The power supply means 16 is an example of a power supply means included in the present invention.

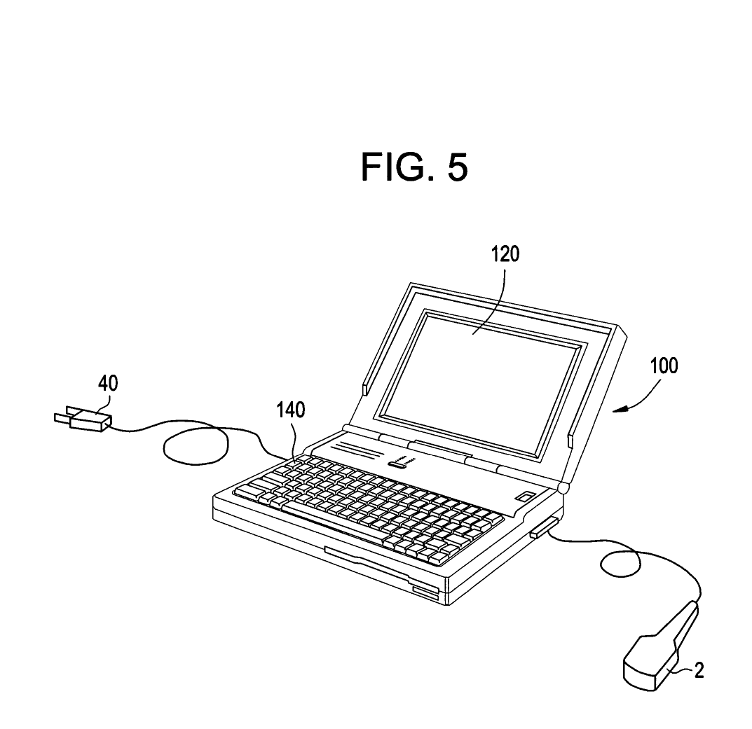

FIG. 5 schematically shows the appearance of the ultrasound diagnostic apparatus. A main body 100 of the apparatus has an appearance analogous to the appearance of a notebook personal computer. One of two panels that can be opened is a graphic display 120 and the other one is a keyboard 140.

The main body 100 includes the foregoing components starting with the transmitting/receiving unit 4 and ending with the battery 18. The ultrasonic probe 2 is connected to the main body 100 over a cable. Furthermore, a mains adaptor 40 can be connected to the main body 100.

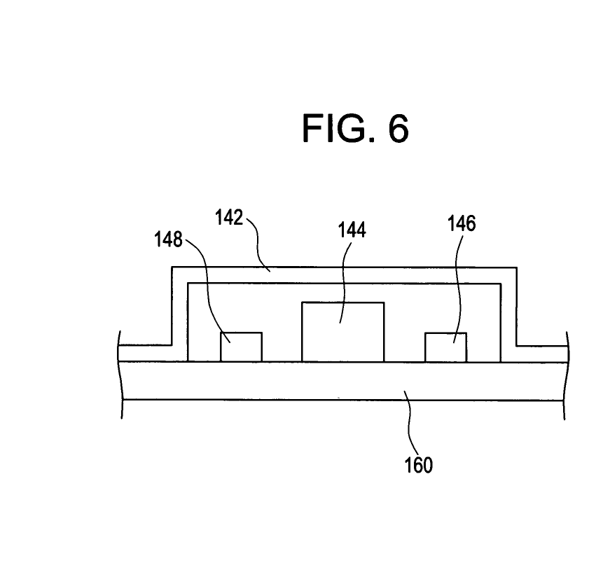

FIG. 6 is a sectional view showing the structure of a key included in the keyboard 140. The key is an example of operation keys included in the present invention. The key has a key top 142. The key top 142 is shaped like a hollow cap made of a translucent elastic material, for example, a plastic. A character, a numeral, a symbol, or the like is inscribed on the apex of the key top 142.

A key switch 144, a light-emitting device 146, and an optical sensor 148 are placed below the key top 142. The key switch 144, light-emitting device 146, and optical sensor 148 are borne by a printed-circuit board 160.

The key switch 144 is a switch that is turned on with the depression of the key top 142. An on or off signal produced responsively to the on or off state of the key switch 144 is transferred to the control unit 12.

The light-emitting device 146 is a light source that lights the key top 142 from below. Illumination light is radiated to outside through the translucent key top 142. Consequently, the degree to which the key is discernible in the dark improves. As the light-emitting device 146, for example, a light-emitting diode is adopted. However, the present invention is not limited to this type of light-emitting device but any other type of light-emitting device will do.

The optical sensor 148 is a sensor that senses light inside the key top 142. Since the key top 142 is translucent, when the light-emitting device 146 does not emit light, ambient light falling through the key top 142 is sensed. As the optical sensor 148, for example, a phototransistor is adopted. However, the present invention is not limited to this type of optical sensor but an appropriate photo-detector will do.

All the keys included in the keyboard 140 have the foregoing structure. Incidentally, the optical sensor 148 need not be included in all of the keys but may be included in plural keys distributed all over the keyboard 140 or in a sole key located at an appropriate position. Otherwise, the optical sensor 148 may be located at an appropriate position on the keyboard 140 instead of being included in any key.

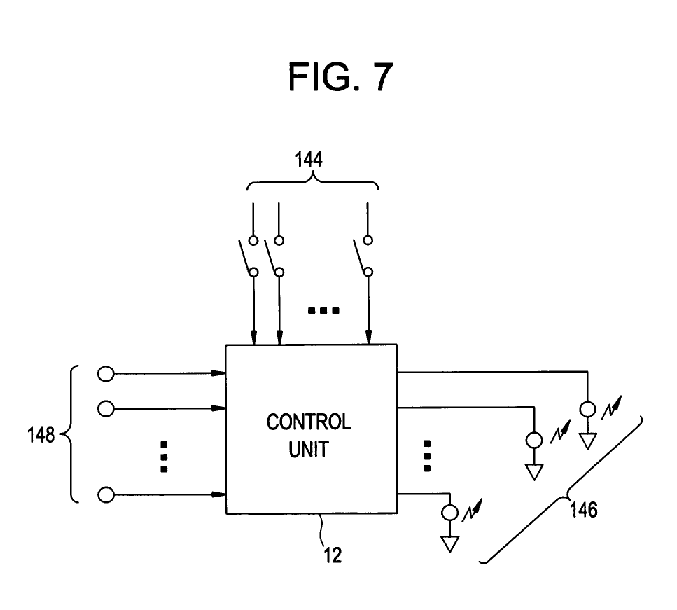

FIG. 7 is a block diagram showing the relationship of the control unit 12 to the key switch 144, light-emitting device 146, and optical sensor 148. An on or off signal produced responsively to the one or off state of the key switch 140 and a light sense signal sent from the optical sensor 148 are transferred to the control unit 12. The control unit 12 provides an output signal so as to control the light-emitting device 146. The control unit 12 is an example of a lighting adjustment means included in the present invention.

The control unit 12 produces a pulsating signal, which has a certain cycle, so as to flicker the light-emitting device. The time interval between adjoining flashings of the light-emitting device is so short that the flicker of the light-emitting device is indiscernible with naked eyes. The time interval is, for example, 20 ms or less. Consequently, the light-emitting device 146 is seen unintermittently lit with naked eyes.

The control unit 12 controls the light-emitting device 146 according to a sense signal which the optical sensor 148 produces at the cyclic timing when the light-emitting device 146 is put out. The sense signal which the optical sensor 148 produces at the timing when the light-emitting device 146 is put out is an ambient-light sense signal. The control unit 12 controls the light-emitting device 146 on the basis of the ambient-light sense signal.

The control unit 12 has a threshold, relative to which an ambient brightness level is determined, set therein. When the sum or mean value of light sense signals produced by all the optical sensors 48 is equal to or smaller than the threshold, the control unit 12 lights the light-emitting devices 146 in a pulsating manner. When the sum or mean value of the light sense signals exceeds the threshold, the light-emitting devices 146 are kept put out.

Consequently, only when an ambient brightness level is equal to or lower than a predetermined level, the keyboard 140 is lit. When the ambient brightness level exceeds the predetermined level, the keyboard is not lit. Consequently, since the keyboard is not lit all the time unlike it is conventionally, the power consumption is reduced accordingly. An operation time permitted when the apparatus is driven by the battery is therefore extended. If the threshold is decreased along with a decrease in a remaining battery capacity, the operation time can be further extended.

When the ambient brightness level exceeds the predetermined level, lighting may not be ceased but the light-emitting devices may be dimmed. Dimming is achieved by reducing the amplitude of a pulsating signal with which each of the light-emitting devices 146 is driven or/and the pulse duration thereof.

When the present apparatus is driven using the mains, the threshold may be made larger than it is when the apparatus is driven with the battery.

Consequently, when the apparatus is driven using the mains, the keyboard is lit in a brighter environment. Otherwise, when the apparatus is driven using the mains, the keyboard may be lit all the time irrespective of the ambient brightness. In either case, since the battery capacity is not consumed, an operating time is left unaffected.

Many widely different embodiments of the invention may be configured without departing from the spirit and the scope of the present invention. It should be understood that the present invention is not limited to the specific embodiments described in the specification, except as defined in the appended claims.

1. An ultrasound diagnostic apparatus with lit operation keys comprising:
a power supply device for switching power to be supplied to the components of the apparatus between power fed from a battery and power fed from the mains; and
a lighting adjustment device for ceasing or minimizing lighting of the keys when a detected ambient brightness level exceeds a predetermined threshold.

2. The ultrasound diagnostic apparatus according to claim 1, wherein the lighting adjustment device lights each of the operation keys by flickering a light source at intervals of a time which is so short that the flicker is indiscernible with naked eyes, and detects an ambient brightness level at the cyclic timing of putting out the light source.

3. The ultrasound diagnostic apparatus according to claim 1, wherein if an ambient brightness level detected when power is fed from the battery exceeds the predetermined threshold, the lighting adjustment device ceases or minimizes lighting of the keys.

4. The ultrasound diagnostic apparatus according to claim 3, wherein the lighting adjustment device decreases the threshold, relative to which a brightness level is determined, along with a decrease in a remaining battery capacity.

5. The ultrasound diagnostic apparatus according to claim 1, wherein when power is fed from the mains, the lighting adjustment device makes the threshold, relative to which a brightness level is determined, larger than when power is fed from the battery.

6. The ultrasound diagnostic apparatus according to claim 1, wherein when power is fed from the mains, the lighting adjustment device lights the operation keys irrespective of an ambient brightness level.
